(19) United States
(12) Patent Application Publication
(10) Pub. No.: US 2024/0118286 A1
(43) Pub. Date: Apr. 11, 2024

(54) METHODS AND SYSTEMS FOR MEASURING ENDOGENOUS ANALYTES UTILZING AN ORIGIN-ADJUSTED APPROACH

(71) Applicant: Laboratory Corporation of America Holdings, Burlington, NC (US)

(72) Inventor: Robert MacNeill, Monroe Township, NJ (US)

(21) Appl. No.: 18/484,368

(22) Filed: Oct. 10, 2023

Related U.S. Application Data
(60) Provisional application No. 63/379,070, filed on Oct. 11, 2022.

(51) Int. Cl. G01N 33/68; G01N 1/18

(52) U.S. Cl. CPC G01N 33/6848; G01N 1/18; G01N 2560/00

(57) ABSTRACT

The field of biomarker detection and drug development is constantly in pursuit of more accurate, reliable, rapid, and inexpensive technology. Methods and systems for quantifying an analyte of interest disclosed herein include steps of obtaining a sample, spiking the sample with the analyte of interest, separating and detecting the sample using LC-MS, and calculating a linear regression of a sample using analytical techniques are described herein. Further, these methods and systems preclude the use and/or calculation of endogenous amounts of analyte present in a sample, yielding to methods and systems that provide increased speed and accuracy for measuring sample analytes.

### RELATED APPLICATIONS

This application claims priority to U.S. Provisional Application No. 63/379,070, filed Oct. 11, 2022. The disclosure of U.S. Provisional Application No. 63/379,070 is incorporated by reference in its entirety herein.

### TECHNICAL FIELD

The present disclosure relates generally to methods and systems for bioanalysis of samples, such as biological specimens.

### BACKGROUND

Accurate and precise quantification of biomarkers is an important objective in the field of diagnostics and drug development. The existing methods to constitute this quantification have barriers to efficiency, practicality, and reliability in that an element of surrogacy must be introduced. This element complicates the approach in the procedure and limits the reliability of the results. Disclosed herein is a new approach for determination of endogenous analyte concentrations through utilization of mass spectrometry and an origin-adjusted approach.

### SUMMARY

Described herein are methods and systems to accurately determine concentrations of endogenous analytes using mass spectrometry (MS). The present disclosure may be embodied in a variety of ways. In certain embodiments, methods and systems include a step of obtaining a sample from a subject. The sample obtained from the subject may be a biosample and may include blood, plasma, serum, sputum, saliva, cerebral spinal fluid, or urine. The biosample may contain one or more analytes that, when determining a presence or a quantity, may be useful in aiding the diagnostics and drug development field. Upon obtaining a biosample, in order to identify a presence of an endogenous analyte of interest, the analyte of interest may first be isolated and/or separated from other analytes in the biosample. Subsequently, the endogenous analyte of interest may be detected using mass spectrometry (MS).

In some examples, a method similar to an analytical standard addition technique may be utilized. Aliquots of the sample then may be spiked with known concentrations of the endogenous analyte of interest such that a linear increase of concentration of the analyte of interest can be realized in the biosample. As a result, the total concentration of the analyte of interest in the biosample will be the sum of the amounts of analyte spiked and the amount of the unknown amount of the analyte of interest. In some examples, the linear increase may be calculated and plotted such that a linear regression formula that includes a slope, may be useful in calculating the endogenous concentration of the analyte of interest in the source of matrix used to construct the calibration. In one particular example, the linear regression formula may exclude the concentration-intercept. The subsequent omission of the x-axis intercept in the linear regression formula may resultantly shift the line over to the origin (0,0). This method can be referred to as the origin-adjusted approach. The linear regression can then be used to accurately interpolate concentration responses of the analyte of interest. This method may be similar to the standard addition approach, yet the origin-adjusted approach simply shifts the endogenously present concentration of the analyte of interest.

Yet, other embodiments are directed to systems comprising a mass spectrometry (MS) analyzer. The MS analyzer may be configured to acquire a total-ion chromatogram for a sample. In one particular embodiment, the MS analyzer may be coupled to an HPLC so that an injected sample may be chromatographically separated from other components in the sample. The separation may be performed using an organic-aqueous solvent gradient and/or a reverse-phase liquid chromatography (LC) column. In some embodiments, the separation of the analyte of interest from other analytes of the sample may be detected via UV irradiation, where, in one example, the wavelength of the energy source may be adjusted to illuminate at a wavelength of 253 nanometers. In one further particular embodiment, the MS analyzer may ionize samples using electrospray ionization (ESI) such that molecules are detected in a positive or a negative, fragmentation mode. Upon detection of the analyte of interest, the MS analyzer may be configured to further provide a chromatogram of absorption to indicate relative concentrations of the analyte of interest plus the spiked concentrations of the analyte of interest. The MS system may be configured to output a report indicating an intensity of fragments of a relative analyte detected and a report indicating the analyte of interest concentration relative to other analytes as an absorption chromatogram.

Further features, advantages and details of the present disclosure will be appreciated by those of ordinary skill in the art from a reading of the figures and the detailed description of the preferred embodiments that follow, such description being merely illustrative of the present disclosure.

Features described with respect with one embodiment can be incorporated with other embodiments although not specifically discussed therewith. That is, it is noted that aspects of the disclosure described with respect to one embodiment, may be incorporated in a different embodiment although not specifically described relative thereto. That is, all embodiments and/or features of any embodiment can be combined in any way and/or combination. The foregoing and other aspects of the present disclosure are explained in detail in the specification set forth below

### BRIEF DESCRIPTION OF THE DRAWINGS

The present disclosure may be better understood with reference to the accompanying drawings, in which embodiments of the methods are shown. This disclosure may, however, be embodied in many different forms and should not be construed as limited to the embodiments set forth herein; rather, these embodiments are provided so that this disclosure will be thorough and complete, and will fully convey the scope of the systems and methods to those skilled in the art. In the drawings, like numbers refer to like elements throughout, and thickness, size and dimensions of some components, lines, or features may be exaggerated for clarity. The order of operations and/or steps illustrated in the figures or recited in the claims are not intended to be limited to the order presented unless stated otherwise. Broken lines in the figures, where used, indicate that the feature, operation, or step so indicated is optional unless specifically stated otherwise. The present disclosure may be better understood by referencing the following non-limiting figures.

FIG. 1 shows a schematic representation of an example of a calibration line shifted towards the origin in accordance with an embodiment of the present disclosure.

FIG. 2 shows a schematic representation of sample preparation of the origin-adjusted approach as compared to a surrogate method and a surrogate analyte approach in accordance with an embodiment of the present disclosure.

FIG. 3 shows chromatograms representative of kynurenine comparing the origin-adjusted approach and the surrogate matrix approach in accordance with an embodiment of the present disclosure. The x-axis is time (minutes), and the y-axis is intensity (counts per second, “cps”).

FIG. 4 shows chromatograms representative of kynurenine comparing the origin-adjusted approach and the surrogate matrix approach in accordance with an embodiment of the present disclosure. The x-axis is time (minutes), and the y-axis is intensity (counts per second, “cps”).

### DETAILED DESCRIPTION

### Terms and Definitions

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “biomarker” is any endogenous biomolecule that may provide biological information about the physiological state of an organism. In certain embodiments, the presence or absence of the biomarker may be informative. In other embodiments, the level of the biomarker may be informative.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer, or section. Thus, a first element, component, region, layer, or section discussed below could be termed a second element, component, region, layer or section without departing from the teachings of the present disclosure. The sequence of operations (or steps) is not limited to the order presented in the claims or figures unless specifically indicated otherwise.

The term “specificity” refers to the ability of the measurement procedure to discriminate the analyte of interest when presented with substances potentially found within a sample. In an embodiment, it is expressed as a percent (%) cross-reactivity and/or response to substances other than analyte of interest in the absence of the analyte of interest.

The term “selectivity” refers to the ability of the measurement procedure to accurately measure the analyte of interest without contribution of the substances potentially found within a sample. In an embodiment, it is expressed as a % cross-reactivity and/or response to substances other than analyte of interest in the presence of the analyte of interest.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Sample” or “patient sample” or “biological sample” or “specimen” are used interchangeably herein. Non-limiting examples of liquid samples that may be used for analysis with the disclosed methods and systems include, blood or a blood product (e.g., serum, plasma, or the like), urine, nasal swabs, skin swabs, lesion swabs, a liquid biopsy sample (e.g., for the detection of cancer), or combinations thereof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue samples or other liquid aspirates. The term “biological sample” further refers to a sample obtained from a biological source, including, but not limited to, an animal, a cell culture, an organ culture, tissue, and the like.

The term “patient” or “subject” is used broadly and refers to an individual that provides a biosample for testing or analysis. The individual “patient” or “subject” whereby a sample is collected, obtained, and/or provided by, includes mammalian subjects such as humans and/or animals.

The terms “purify” or “separate” or derivations thereof do not necessarily refer to the removal of all materials other than the analyte(s) of interest from a sample matrix. Instead, in some embodiments, the terms “purify” or “separate” refer to a procedure that enriches the amount of one or more analytes of interest relative to one or more other components present in the sample matrix. In some embodiments, a “purification” or “separation” procedure can be used to remove one or more components of a sample that could interfere with the detection of the analyte, for example, one or more components that could interfere with detection of an analyte by mass spectrometry

As used herein, the term “chromatography” refers to a process in which a chemical mixture carried by a liquid or gas is separated into components as a result of differential distribution of the chemical entities as they flow around or over a stationary liquid or solid phase.

The term “Lower Limit of Quantitation” (LLOQ) refers to the lowest amount of analyte in a sample that can be quantitatively determined with stated acceptable precision and accuracy.

The term “Upper Limit of Quantitation” (ULOQ) refers to the highest amount of analyte in a sample that can be quantitatively determined without dilution.

The term “Intra-run Imprecision” refers to the closeness of the agreement between the results of successive measurements of the same measurand carried under the same conditions of measurements (same analytical run).

The term “Inter-run Imprecision” refers to the closeness of the agreement between independent test results obtained under stipulated conditions (different analytical runs and/or operators, laboratories, instruments, reagent lots, calibrators, etc.).

The term “Maximum Dilution/Concentration” refers to the established laboratory specifications for the maximum dilution and/or concentration that may be performed to obtain a reportable numeric result.

The term “Reference Interval” refers to an interval that, when applied to the population serviced by the laboratory, correctly includes most of the subjects with characteristics similar to the reference group and excludes the others.

As used herein, “liquid chromatography” (LC) refers to a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). “Liquid chromatography” includes reverse phase liquid chromatography (RPLC), high performance liquid chromatography (HPLC) and high turbulence liquid chromatography (HTLC). As used herein, the term “HPLC” or “high performance liquid chromatography” refers to liquid chromatography in which the degree of separation is increased by forcing the mobile phase under pressure through a stationary phase, typically a densely packed column.

The chromatographic column typically includes a medium (i.e., a packing material) to facilitate separation of chemical moieties (i.e., fractionation). The medium may include minute particles. The particles can include a bonded surface that interacts with the various chemical moieties to facilitate separation of the chemical moieties such as the biomarker analytes quantified in the experiments herein. One suitable bonded surface is a hydrophobic bonded surface such as an alkyl bonded surface. Alkyl bonded surfaces may include C-4, C-8, or C-18 bonded alkyl groups, preferably C-18 bonded groups. The chromatographic column includes an inlet port for receiving a sample and an outlet port for discharging an effluent that includes the fractionated sample. In the method, the sample (or pre-purified sample) may be applied to the column at the inlet port, eluted with a solvent or solvent mixture, and discharged at the outlet port. Different solvent modes may be selected for eluting different analytes of interest. For example, liquid chromatography may be performed using a gradient mode, an isocratic mode, or a polytyptic (i.e. mixed) mode.

The term “high turbulence liquid chromatography” or “HTLC” or “turbulent flow liquid chromatography” or “TFLC” analysis relies on column packings in which turbulent flow of the sample through the column is the basis for separation of the analyte of interest from the sample. In such columns, separation is a diffusional process. Turbulent flow, such as that provided by HTLC columns and methods, may enhance the rate of mass transfer, improving the separation characteristics provided. For example, in a typical high turbulence or turbulent liquid chromatography system, the sample may be injected directly onto a narrow (e.g., 0.5 mm to 2 mm internal diameter by 20 to 50 mm long) column packed with large (e.g., >25 micron) particles. When a flow rate (e.g., 3-500 mL per minute) is applied to the column, the relatively narrow width of the column causes an increase in the velocity of the mobile phase. The large particles present in the column can prevent the increased velocity from causing back pressure and promote the formation of vacillating eddies between the particles, thereby creating turbulence within the column.

In high turbulence liquid chromatography, the analyte molecules can bind quickly to the particles and typically do not spread out, or diffuse, along the length of the column. This lessened longitudinal diffusion typically provides better, and more rapid, separation of the analytes of interest from the sample matrix. Further, the turbulence within the column reduces the friction on molecules that typically occurs as they travel past the particles. For example, in traditional HPLC, the molecules traveling closest to the particle move along the column more slowly than those flowing through the center of the path between the particles. This difference in flow rate causes the analyte molecules to spread out along the length of the column. When turbulence is introduced into a column, the friction on the molecules from the particle is negligible, reducing longitudinal diffusion.

As used herein, the term “analytical column” refers to a chromatography column having sufficient chromatographic plates to effect a separation of the components of a test sample matrix. Preferably, the components eluted from the analytical column are separated in such a way to allow the presence or amount of an analyte(s) of interest to be determined. In some embodiments, the analytical column comprises particles having an average diameter of about 5 μm. In some embodiments, the analytical column is a functionalized silica or polymer-silica hybrid, or a polymeric particle or monolithic silica stationary phase, such as a phenyl-hexyl functionalized analytical column.

Analytical columns can be distinguished from “extraction columns,” which typically are used to separate or extract retained materials from non-retained materials to obtained a “purified” sample for further purification or analysis. In some embodiments, the extraction column is a functionalized silica or polymer-silica hybrid or polymeric particle or monolithic silica stationary phase, such as a Poroshell SBC-18 column.

As used herein, the terms “mass spectrometry” or “MS” generally refer to methods of filtering, detecting, and measuring ions based on their mass-to-charge ratio, or “m/z.” In MS techniques, one or more molecules of interest are ionized, and the ions are subsequently introduced into a mass spectrometer where, due to a combination of electric fields, the ions follow a path in space that is dependent upon mass (“m”) and charge (“z”).

In certain embodiments, the mass spectrometer uses a “quadrupole” system. In a “quadrupole” or “quadrupole ion trap” mass spectrometer, ions in an oscillating radio frequency (RF) field experience a force proportional to the direct current (DC) potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

In certain embodiments, “tandem mass spectrometry” (MS/MS) is used. Tandem mass spectrometry (MS/MS) is the name given to a group of mass spectrometric methods wherein “parent or precursor” ions generated from a sample are fragmented to yield one or more “fragment or product” ions, which are subsequently mass analyzed by a second MS procedure. MS/MS methods are useful for the analysis of complex mixtures, especially biological samples, in part because the selectivity of MS/MS can minimize the need for extensive sample clean-up prior to analysis. In an example of an MS/MS method, precursor ions are generated from a sample and passed through a first mass filter (quadrupole 1 or Q1) to select those ions having a particular mass-to-charge ratio. These ions are then fragmented, typically by collisions with neutral gas molecules in the second quadrupole (Q2), to yield product (fragment) ions which are selected in the third quadrupole (Q3), the mass spectrum of which is recorded by an electron multiplier detector. The product ion spectra so produced are indicative of the structure of the precursor ion, and the two stages of mass filtering can eliminate ions from interfering species present in the conventional mass spectrum of a complex mixture.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “electron ionization” as used herein refers to methods in which an analyte of interest in a gaseous or vapor phase interacts with a flow of electrons. Impact of the electrons with the analyte produces analyte ions, which may then be subjected to a mass spectrometry technique. The term “chemical ionization” as used herein refers to methods in which a reagent gas (e.g. ammonia) is subjected to electron impact, and analyte ions are formed by the interaction of reagent gas ions and analyte molecules.

The term “electrospray ionization,” or “ESI,” as used herein refers to methods in which a solution is passed along a short length of capillary tube, to the end of which is applied a high positive or negative electric potential. Upon reaching the end of the tube, the solution may be vaporized (nebulized) into a jet or spray of very small droplets of solution in solvent vapor. This mist of droplet can flow through an evaporation chamber which is heated slightly to prevent condensation and to evaporate solvent. As the droplets get smaller the electrical surface charge density increases until such time that the natural repulsion between like charges causes ions as well as neutral molecules to be released.

The term “Atmospheric Pressure Chemical Ionization,” or “APCI,” refers to mass spectroscopy methods that are similar to ESI, however, APCI produces ions by ion-molecule reactions that occur within a plasma at atmospheric pressure. The plasma is maintained by an electric discharge between the spray capillary and a counter electrode. Then, ions are typically extracted into a mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “Atmospheric Pressure Photoionization” (“APPI”) refers to the form of mass spectroscopy where the mechanism for the photoionization of molecule M is photon absorption and electron ejection to form the molecular M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Drug compounds in protic solvents are usually observed as MH+, whereas nonpolar compounds such as naphthalene or testosterone usually form M+ (see e.g., Robb et al., 2000, Anal. Chem. 72(15): 3653-3659).

The terms “calibration line,” “calibration curve,” “standard curve,” and “standard line,” as used, may be used interchangeably to describe either an internal or an external linear response as a function of concentration of an analyte of interest. Amount of the analyte of interest added may vary incrementally. Amount added may or may not be known initially. A calibration line may be generated as a trend line from multiple coordinates of response as a function of increasing concentration of the analyte of interest. The mathematical formula for the calibration line may be used for interpolation of responses.

The term “on-line” refers to purification or separation steps that are performed in such a way that the test sample is disposed, e.g., injected, into a system in which the various components of the system are operationally connected and, in some embodiments, in fluid communication with one another.

In contrast to the term “on-line”, the term “off-line” refers to a purification, separation, or extraction procedure that is performed separately from previous and/or subsequent purification or separation steps and/or analysis steps. In such off-line procedures, the analytes of interests typically are separated, for example, on an extraction column or by liquid/liquid extraction, from the other components in the sample matrix and then collected for subsequent introduction into another chromatographic or detector system. Off-line procedures typically require manual intervention on the part of the operator.

As used herein, the terms “spike,” “spiked,” “spiking,” are used to describe a physical process of adding a known concentration of an analyte to a sample containing an unknown concentration of the same analyte, where the unknown concentration of the same analyte is considered to be the endogenous concentration of a sample. Spiking may be done at a desirable concentration, but can typically be done in consistent, incremental additions such that when plotted on a concentration-response graph, the spiked concentrations generate a linear, or close to linear curve.

### Methods for Measuring Endogenous Analytes

The present invention will now be described more fully hereinafter, in which embodiments of the invention are shown. This invention may, however, be embodied in different forms and should not be construed as limited to the embodiments set forth herein. Rather, these embodiments are provided so that this disclosure will be thorough and complete, and will fully convey the scope of the systems and methods to those skilled in the art.

Described herein are novel methods including an assay technique for determining a presence or an amount of a certain endogenous analyte of interest in a sample, such as a biological matrix. Finding methods and systems that may provide a simple and reliable manner of quantifying endogenous analytes in a sample is a goal in the field of biomarker quantification. While methods such as a surrogate analyte, surrogate matrix, and standard addition approach may be useful, these methods may be limited in that a concentration of already-present, endogenous analyte must be accounted for, which can lead to misleading results if parallelism is not or cannot be established and endogenous concentrations are erroneous. A method for circumventing such calculations may be useful in determining accurate concentrations of an analyte of interest.

In many examples, an origin-adjusted (OA) approach may be based on a standard addition technique in which a biological matrix is spiked with the endogenous analyte of interest to create a standard curve. Following calculations, the OA approach calculates the x-axis or concentration axis intercept of zero, termed the concentration-intercept. This concentration-intercept is calculated such that the x-intercept is set to zero, thus providing a standard curve that is anchored at the origin.

In some embodiments, utilizing the OA approach may be preclude the use of alternative methods for measuring analytes of interest not limited to surrogate analyte, surrogate matrix, and standard addition approach. Further, OA approach embodiments may preclude parallelism from external calibration for comparison.

Certain embodiments may contain an adjustment to a line of standard addition construction that may be supported by linear regression characteristics of bioanalytical LC-MS methods. Such embodiments may contain a linear regression that can be embodied by the formula y=ax+b where y and x are a point's given coordinates, b is the y-intercept, and where a is the slope of the line. Much like a xenobiotic method, some examples may have a small margin of natural and innate instrumental error at lower concentrations. In particular, the line may intercept the origin at (0,0) with as little error as possible.

Other embodiments, however, utilize the OA approach through an adjustment of the line such that a shift of the calculated line, with the same slope, begins at the intercept of the origin. Further, this embodiment utilizes a linear regression line formula without the term “+b” such that the new formula is represented as such with an x-axis intercept of 0: y=ax.

In these embodiments, because the magnitude of the concentration-intercept is representative of the endogenous level, the line shift to intersect the origin may be viewed as analogous to the removal of the endogenous concentration. This may be useful as the newly shifted line, precluding endogenous concentrations, may provide increased accuracy.

In an embodiment, the shifted calibration line may be more accurate since the slope of the calibration is representative of that of the unadulterated molecular analyte in authentic matrix and the origin intercept is expected for a non-endogenous analyte. A calibration line may be less linear at higher concentrations than at lower concentrations.

In some embodiments, methodologies relating to similar principles may be run in parallel to support precision and reliability from comparative tests. Such methodologies may include the surrogate analyte, surrogate matrix, and standard addition approach.

In some embodiments, the calculated amount of each analyte in each sample may be determined by comparison of the sample response or response ratio when employing internal standardization to calibration curves generated by spiking a known amount of purified analyte material into a standard test sample. In one embodiment, calibrators are prepared at known concentrations to generate a response or response ratio when employing internal standardization versus concentration in a calibration curve. In an embodiment, this determination is performed at least in part by a computer or data analysis system and/or a non-transitory computer readable storage medium containing instructions which, when executed on the one or more data processors, cause the one or more data processors to perform actions to make this determination.

Some embodiments may begin a method of detecting endogenous analytes of interest through first detecting a presence of the analyte or analytes of interest in a sample obtained from a subject. The detecting of the presence of the analyte of interest may be performed by mass spectrometry (MS) and by other methods of mass detection as well. Upon molecular fragmentation of the analyte of interest, the fragments may be further fragmented in the instrument for further confirmation of a certain mass. Secondary fragmentation, or MS-MS, further known as tandem MS, may be useful in determining the presence of the analyte of interest although the presence of the analyte of interest may be determined without tandem MS.

In some embodiments, the endogenous analyte of interest may be extracted, isolated, separated, and/or purified from other analytes present in a certain sample. This separation may follow detection or may precede detection. Such separation may be performed by a liquid chromatography instrument at high-pressure, using a reverse-phase column at various flow rates, as well as by other methods. Other methods may be manual or automated.

In some embodiments, a sample of biological origin, obtained from a subject, may be used for study. The sample may be a tissue, hair, or bodily fluid. In one embodiment, a sample of blood, containing the endogenous analyte of interest, may be the sample used for study. An analyte contained within the blood sample may vary accordingly but may generally include low- and high-molecular weight organic compounds such as amino acids and proteins, respectively. In one example of the present disclosure, the sample may be analyzed for the presence of tryptophan and kynurenine. Sample preparation may involve liquid-solid filtration and/or liquid-liquid extraction. In some embodiments, the sample may directly be injected into an instrument for analysis. In such embodiments, the instrument may be a high turbulence liquid chromatography system. Following sample preparation and containment, the sample may be analyzed using a mass spectrometry (MS) system for determining a presence of the analyte of interest within the sample. A sample that contains the endogenous analyte of interest, as determined by MS, or MS-MS fragmentation intensity, may further be isolated using an analytical liquid chromatography system paired to the MS system. Upon isolation, an absorption chromatogram may output to report an amount of the analyte of interest present.

Further, any of the previous embodiments of the present disclosure may include adding a known concentration of the endogenous analyte of interest to multiple samples for analysis, otherwise known as “spiking” the sample. The known concentrations of the endogenous analyte of interest may be added equally incrementally across several samples such that a pattern of additions may be generated. An example of the pattern, where m is the endogenous analyte and the concentration is added numerically, is m, m+1, m+2, m+3 . . . m+n. The spiking of the sample may aid in generating a linear calibration curve for interpolation such that the total amount of the analyte of interest present in a sample is the sum of the endogenous amount of the analyte of interest and the known, added concentrations of the analyte of interest. Adding a known concentration of the analyte of interest may provide a linear increase in concentration, as determined by the absorption chromatogram. Using the concentration-response of the spiked analyte of interest, a line can be plotted on a graph with concentration on the x-axis and concentration-response on the y-axis. Concentration-response may be an absorbance of a concentration of analyte or other like responses. Using the concentration and the respective response, a line may be plotted. In some embodiments, the plotted line, at the x-intercept, may be translated to intercept the origin such that the x-intercept is equal to zero. Translation of the line may be referred to as the origin-adjusted line. The linear curve may then be used to interpolate other concentrations from a response using a respective mathematical formula for the linear calibration curve.

An alternative embodiment may utilize preparatory high-pressure liquid chromatography (PHPLC) or analytical high-pressure liquid chromatography (HPLC). The separation of the analyte of interest from the whole biosample or otherwise from other analytes, may be performed on PHPLC or HPLC prior to or preceding detection with MS.

in other embodiments, ultra-high pressure/ultra-high performance liquid chromatography may be utilized as the system for separation and analysis of the endogenous analyte of interest. Similarly, other forms and/or combinations of liquid chromatography may be utilized as an embodiment for the present disclosure.

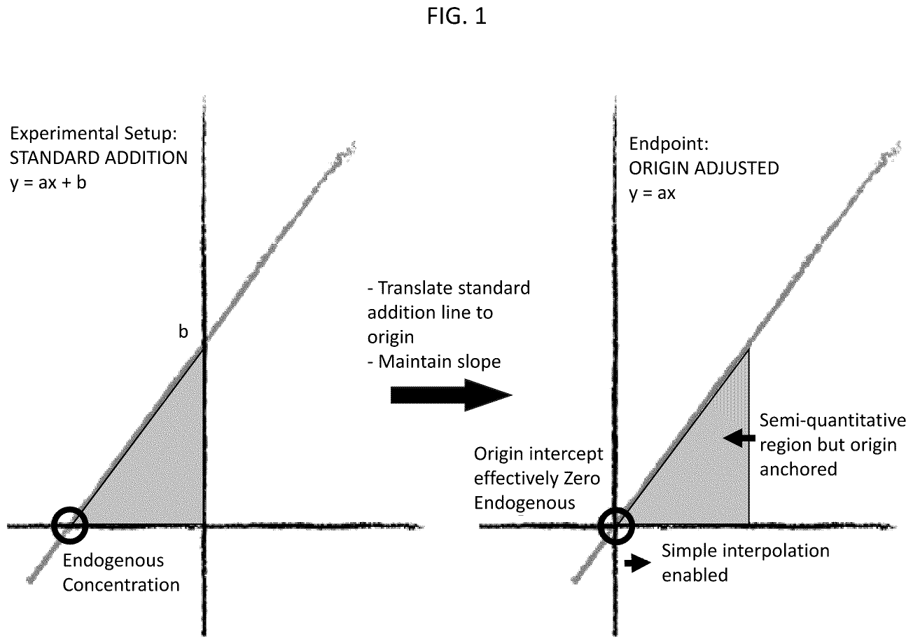

FIG. 1 shows a schematic representation of an example of a calibration curve shift of a method of the present disclosure. According to one example, the plot on the left indicates a graph representative of a standard addition calibration curve plotted as a result of calculating. In an embodiment, a shaded region below a point demarked as “b” may indicate a semi-quantitative region. The semi-quantitative region may be representative of the response to concentrations below a lowest calibration point. The semi-quantitative region may be further outlined by the x-intercept which may indicate an endogenous concentration of the analyte of interest. In an embodiment, the semi-quantitative region is further outlined by the origin at the intersection of the x and y-axis. A subsequent shift, directionally, in a positive x-axis direction, such that the endogenous concentration-intercept is translated to the x and y-axis, may depict an embodiment of the present disclosure.

In an embodiment of the present disclosure, a right plot may be considered an Origin-Adjusted Approach plot. A plot may be adjusted directionally in a positive or negative, x or y-axis direction. In one embodiment, the calibration line is adjusted and translated on the x-axis. The semi-quantitative region may also be translated accordingly, with the calibration line. The endogenous concentration-intercept may then be anchored at the x and y-axis intersection. Further, respective concentrations from the calibration line may also be shifted such that the point demarked as “b” may be plotted on the positive, x-axis.

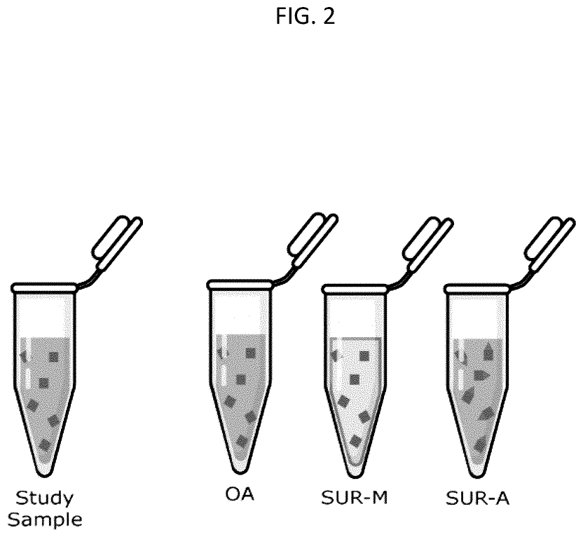

FIG. 2 further shows a schematic representation of an example of a method of the present disclosure. In comparing various methods to the origin-adjusted approach, sample preparation may vary accordingly. The OA approach container may contain an identical matrix and the analyte of interest similar to a container of the study sample, thereby reducing inaccuracy associated with lack of parallelism. In one embodiment, a surrogate approach may contain an analyte of interest, analogous to a study sample, but may be suspended in a matrix different from the study sample. Alternatively, in another example, a surrogate approach may also contain a matrix similar to the one of the study sample, yet may contain a surrogate analyte. Both surrogate approaches may serve to generate a calibration curve for further detection of analytes yet may provide erroneous results from endogenous analyte calculations. In an embodiment, the OA approach sample preparation and make up may be similar to the study sample to reduce innate uncertainty of the surrogate approaches.

### Systems for Measuring Endogenous Analytes

Also disclosed are systems for performing any of the steps of the disclosed methods and computer-implemented instructions for performing any of the steps of the disclosed methods or running any of the parts of the disclosed systems.

For example, disclosed is a system comprising one or more stations or components for performing any of the previous method embodiments. In one embodiment of the present disclosure, a system may comprise a station for detecting an analyte of interest, a station for chromatographically separating the analyte of interest from other components in the sample, and a station for analyzing the chromatographically separated analyte of interest by mass spectrometry to determine the presence or amount of the analyte of interest in the sample.

Also disclosed is a computer-program product tangibly embodied in a non-transitory machine-readable storage medium including instructions configured to cause one or more data processors to perform any of the steps of the disclosed methods or to run any of the components of the disclosed systems. For example, in certain embodiments, disclosed is a computer-program product tangibly embodied in a non-transitory machine-readable storage medium including instructions configured to cause one or more data processors to perform processing comprising: (a) detecting an analyte of interest, (b) separating the analyte of interest from the sample, and (c) detecting a presence or amount of the analyte of interest separated from the sample.

As will be appreciated by one of skill in the art, the items of the present disclosure may be embodied as an apparatus, a method, data or signal processing system, or computer program product. Accordingly, the items of the present disclosure may take the form of an entirely software embodiment, or an embodiment combining software and hardware aspects. Furthermore, certain embodiments of the present disclosure may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, but is not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium, upon which the program is printed, as the program can be electronically captured, via, for instance, optical scanning of the paper or other medium, then compiled, interpreted, or otherwise processed in a suitable manner if necessary, and then stored in a computer memory.

Computer program code for carrying out operations of the present disclosure may be written in an object-oriented programming language such as java7, Smalltalk, Python, Labview, C++, or VisualBasic. However, the computer program code for carrying out operations of the present disclosure may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

In some embodiments, a mass spectrometry system may be used for filtering, detecting, and measuring ions based on their mass-to-charge ratio. In MS, one or more molecules of interest may be ionized, and the ions may be subsequently introduced into a mass spectrometer where, due to a combination of electric fields, the ions may follow a path in space that is dependent upon mass (“m”) and charge (“z”).

In some embodiments, the mass spectrometer system may further utilize a quadrupole system. In a quadrupole or quadrupole ion trap mass spectrometer, ions in an oscillating radio frequency (RF) field may experience a force proportional to the direct current (DC) potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

In some embodiments, tandem mass spectrometry system (MS/MS or MS-MS) may be used. Tandem mass spectrometry may be defined as when parent or precursor ions generated from a sample are fragmented to yield one or more fragment or product ions, which may be subsequently mass analyzed by a second MS procedure.

In some embodiments, separation of the analyte of interest from other analytes may be performed using liquid chromatography (LC) where a process of selective retardation of one or more components of a fluid solution may occur as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). LC may include reverse phase liquid chromatography (RPLC), high performance liquid chromatography (HPLC) and high turbulence liquid chromatography (HTLC). Further, chromatography may refer to a process in which a chemical mixture carried by a liquid or gas may be separated into components as a result of differential distribution of the chemical entities as they flow around or over a stationary liquid or solid phase.

In certain embodiments, a chromatographic column may include a medium to facilitate separation of chemical moieties. The medium may include minute particles. The particles can include a bonded surface that interacts with the various chemical moieties to facilitate separation of the chemical moieties such as the biomarker analytes quantified in the experiments herein. One suitable bonded surface may be a hydrophobic bonded surface such as an alkyl bonded surface. Alkyl bonded surfaces may include C-4, C-8, or C-18 bonded alkyl groups, preferably C-18 bonded groups.

Therefore, in some embodiments, the analyte of interest may be extracted, isolated separated, and/or purified from other analytes present in a certain sample. This separation may follow detection or may precede detection. Such separation may be performed by a liquid chromatography instrument at high-pressure, using a reverse-phase column at various flow rates, as well as by other methods. Other methods may be manual or automated.

In some embodiments, the system may be a liquid chromatography system paired with a mass spectrometry system configured to separate and detect the analyte of interest. Certain embodiments may be directed toward a first step of determining and/or detecting the presence of the analyte of interest using the system while sequentially performing a step of chromatographically separating, isolating, and/or purifying the analyte of interest from the rest of a sample. Alternatively, some embodiments may have a step of first chromatographically separating the analyte using the system, followed by a step detecting the analyte with the system. In some embodiments, spiking the sample with the analyte of interest may be performed prior to insertion of the sample into the system.

In some embodiments, the system may be configured to provide a user with a concentration-response to the analyte of interest in a sample. The system may further be configured to calculate a linear regression and adjust the concentration-intercept of the linear regression by translating the linear regressing towards the positive x-axis. In some embodiments, a final step of a system may include determining a quantity of the analyte of interest in a sample using the adjusted concentration-intercept. A report may be output from a system with a detailed analysis of the concentration-response plot.

### Examples

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

An example of a method for examining a sample from a subject is described herein. An example application to show the validity of the origin-adjusted (OA) approach is based on a dual analyte assay in the field of biomarkers, tryptophan and kynurenine in plasma which has been reported by a surrogate analyte (SUR-A) and a surrogate matrix (SUR-M) assay. The SUR-M and SUR-A approaches, both involving MS, also involve the use of response factors (RF) to account for response differences, hence the calibration curve slope differences between the unlabeled analytes and the surrogate analytes. In some instances, correction of the response differences may lead to a lack of reliability of a method or an approach. Parallelism between calibration curves of one assay approach to a second assay approach or a reference curve may be helpful in the uniformity of linear regression characteristics, particularly the slope, to validly represent a genuine analyte's response-concentration characteristics, and this is ideally done without manual intervention. Parallelism may be useful in determining the accuracy of results from a surrogate method as compared to the OA approach method, particularly in defining the slope. While some approaches such as the SUR-M and the SUR-A methods typically employ parallelism as a technique to ensure accuracy, the OA approach precludes such a technique, thereby providing increased advantages over other approaches.

An initial phase of testing involved a comparison of SUR-A, SUR-M, and OA approach sample preparation and analysis for both analytes, tryptophan and kynurenine, working towards generating fair comparisons for a method of quantification of two analytes from the kynurenine pathway. The scheme involves kynurenine (0.0250 to 2.50 μg/mL) and tryptophan (0.500 to 50.0 μg/mL) quantified in human plasma via HPLC and tandem mass spectrometric detection. Chromatographic separation with was performed on Shimadzu 20-Series HPLC systems with associated mass spectrometric detection performed on Sciex 5500 instruments in multiple reaction monitoring (MRM) mode.

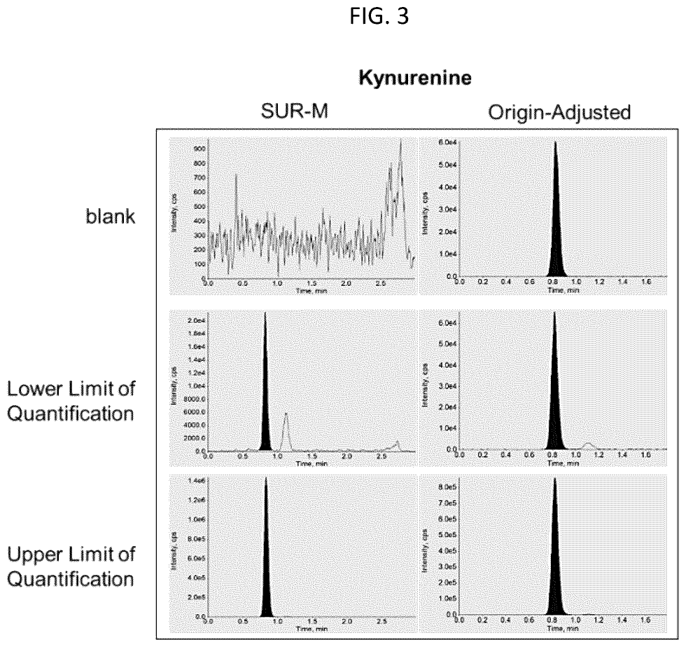

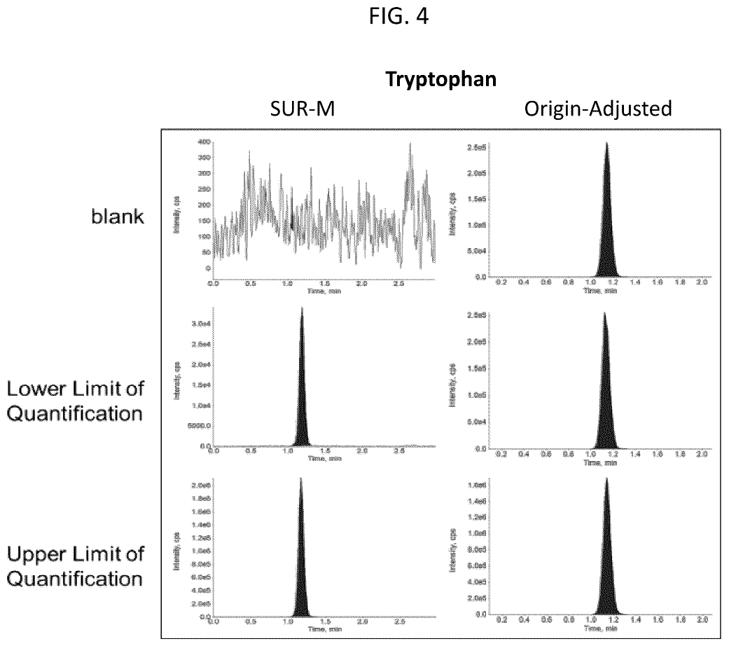

FIG. 3 and FIG. 4 provide an example of total ion chromatograms from a sample of kynurenine and tryptophan, respectively, as a result of the surrogate matrix approach and the OA approach according the one embodiment of the present disclosure. The left block of plots displays kynurenine chromatograms while the right block displays tryptophan chromatograms. As can be observed in the chromatograms for both analytes, the blank, compared to the origin-adjusted chromatograms, display only “noise” since there is no analyte present in the matrix. Conversely, in the OA approach, the analyte is present in the blank chromatograms similar to the chromatograms of LLOQ. Chromatograms of the ULOQ further display comparable intensities of both analytes, resulting from the surrogate matrix and the origin-adjusted approach.

The first surrogate analyte was a ¹⁵N₂-labeled isotopologue for kynurenine while the associated internal standard, in all approaches, was a D4-kynurenine. The second surrogate analyte was a ¹⁵N₂-labeled isotopologue for tryptophan while the associated internal standard, in all approaches, was a D5-tryptophan. As surrogate analytes, isotopologues provided an increase in mass of at least two atomic mass units while internal standards provided an increase in mass of four atomic mass units. The surrogate matrix used was 0.2% formic acid (aq). OA approach calibrants were in genuine matrix, for SUR-A, and genuine unlabeled analytes, as per SUR-M, thereby embracing all authenticity available. The results showed that indeed there were correction factors that would be useful for SUR-A in that the slopes calculated, for both analytes, manifestly did not agree with the slopes from the OA approach and SUR-M approaches. Meanwhile the slopes from the latter two approaches did correlate convincingly with each other. The data are shown in Table 1. This experiment was repeated on two more occasions for confirmation.

TABLE 1
Initial test results comparing kynurenine
slopes from SUR-A, SUP-M and OA.
Kynurenine

| | Slope data (arbitrary units) | | |
|---|---|---|---|
| | SUR-A | SUR-M | OA |
| 1st occasion | 23.7 | 34.9 | 37.5 |
| 2nd occasion | 18.9 | 29.6 | 33.8 |

Therefore, in consideration of the response difference for SUR-A and understanding of the slope and how it relates to parallelism, it was clear the only option in this work was to use SUR-M as the comparator approach. This holds the additional ideal of utilizing a common approach for biomarker detection.

The work included, for both analytes, an initial numerical comparison of the slopes generated from six separate analytical occasions, then for the latter analytical batch in the sequence, the two sets of quality control sample concentrations interpolated through the SUR-M line and the OA approach line are compared. The quality control samples are all prepared in the SUR-M manner.

Table 2 shows the data for the slope and interpolated quality control sample comparisons. In a manner aligned with the slope test described immediately prior, the results of this test also strongly indicate that the OA approach, for both analytes, is equivalent to SUR-M. For each analyte, the quality control samples, prepared in the SUR-M manner, have peak area ratio responses interpolated through the SUR-M line complete with intercept. Resultantly, the sample responses being interpolated through the OA approach line, with the slope generated from its own calculation and the intercept simply set to zero, yields the Origin-Adjustment. For kynurenine, the percent difference never exceeds a magnitude of 13.8%, which is seen at the lower limit of quantification (LLOQ), and the overall average difference over the six analytical nominal concentrations is 0.137%. For tryptophan, the difference never exceeds a magnitude of 8.37%, which again is seen at the LLOQ, and the overall average difference over the six analytical nominal concentrations is 2.05%. The LLOQ showing the most difference in both cases is likely an artifact of the closest proximity to the origin and where the imprecision is highest for the extrapolative approach.

TABLE 2
Percent Differences of QC Samples Interpolated
through SUR-M and OA Calibration Lines

| Kynurenine | | | Tryptophan | | |
|---|---|---|---|---|---|
| | SUR-M | OA | | SUR-M | OA |
| Slope | 32.3 | 34.8 | Slope | 2.20 | 2.37 |
| Intercept | 0.166 | Origin | Intercept | 0.0404 | Origin |
| | % | | | % | |
| (n = 6) | difference | | (n = 6) | difference | |
| LLOQ QC | 13.8 | | LLOQ QC | 8.37 | |
| Low QC | −0.338 | | Low QC | −2.33 | |
| Mid QC | −6.83 | | Mid QC | −7.00 | |
| ULOQ QC | −7.18 | | ULOQ QC | −7.25 | |
| Mean | −0.137 | | Mean | −2.05 | |

Table 3 shows the data resultant from the multi-batch slope comparisons, where the slope, as described, is a component for reliable calculation of sample concentrations. For both analytes, the percent differences are demonstrative of equivalence between SUR-M and OA. In the case of kynurenine, the difference never exceeds a magnitude of 13.2%, and the overall average difference over the six analytical occasions is −6.05%. In the case of tryptophan, the data are even more convincing. The difference never exceeds a magnitude of 8.56%, and the overall average difference over the six analytical occasions is −1.63%. These numbers speak for themselves in the context of typical bioanalytical acceptance criteria for percent difference, firmly established at within +/−15% at the most severe.

Referring again to FIG. 1, it may readily be observed that there exists a semi-quantitative region in the calibrations, as denoted by the shaded area. This region is below the point of lowest calibrant peak area, hence lacks complete characterization in the same way that standard addition extrapolates from the origin to the intercept. The region extends to zero, the origin. In the way it is used, the origin acts as an anchor point. Therein, the line anchored to the origin through the undefined region between itself and the lowest calibrant peak area responses precludes the imprecision encountered with the lower concentration calibrants. Precision is poorest at the lowest concentrations, and this is directly linked to the nature of spiking the calibrants atop an existing endogenous level, in conjunction with the heteroscedasticity of the concentration-response relationship. The inherent response variability observed for a given nominal concentration calibrant sample is in reality that associated with the endogenous in addition to the over-spiked nominal, more variability than in a pharmacokinetic assay calibration and which will worsen with increasing underlying endogenous. It will also affect the lower concentration calibrants more than higher. Importantly, in addition to the anchor confronting this calibrant effect, the added imprecision would not be associated with real study samples as they will not have been over-spiked prior to analysis thus their response variability will not deviate from that of their own innate levels.

TABLE 3
Slope percent differences between OA and SUR-M.

| | Kynurenine | | | Tryptophan | | |
|---|---|---|---|---|---|---|
| | Calculated slope values | | | Calculated slope values | | |
| | | | % | | | % |
| | SUR-M | OA | difference | SUR-M | OA | difference |
| Batch 1 | 29.1 | 32.8 | 12.0 | 1.46 | 1.45 | −0.687 |
| Batch 2 | 34.8 | 39.7 | 13.2 | 1.90 | 2.02 | 6.12 |
| Batch 3 | 41.4 | 42.2 | 1.91 | 2.07 | 1.90 | −8.56 |
| Batch 4 | 42.8 | 43.5 | 1.62 | 1.93 | 1.95 | 1.03 |
| Batch 5 | 43.6 | 43.7 | 0.229 | 1.99 | 2.08 | 4.42 |
| Batch 6 | 32.3 | 34.8 | 7.45 | 2.20 | 2.37 | 7.44 |
| Average | 37.3 | 39.5 | | 1.93 | 1.96 | |
| Mean % | | 6.05 | | | 1.63 | |
| difference | | | | | | |

In further, regarding the semi-quantitative region, it can be readily acknowledged that biomarker levels can vary in either direction, naturally, but must also be considered unlikely to take a drastic drop below established endogenous. Furthermore, having sample levels impinge on the semi-quantitative region is unlikely to affect a clinical outcome. It can also be borne in mind that, in this region, if an empirical degree of concentration-response characterization is ever called for, it can be done in solution or surrogate scenario. Then, it is also worth acknowledging that non-linearity or curvature effects in LC-MS or LC-MS/MS typically will not be manifest in lower concentration regions, but rather at higher concentrations. Imprecision may be encountered, synonymous with matrix effect, which may sometimes seem like curvature but in reality, is not. Again, the origin anchor helps in this regard.

It remains true that the validity of the origin intercept and furthermore the results obtained through OA approach are as innately reliable as the endogenous concentrations calculated through standard addition where the intercept is utilized to this end, via extrapolation. The origin anchor is as valid as the reliability of standard addition calculated concentrations, known to be reliable despite the element of extrapolation. In a well-designed calibration scheme, the characterization of the calibration line afforded from above the endogenous level is sufficient for reliability.

Thus, a novel multi-advantageous approach, referred to as Origin-Adjusted (OA) approach, for the determination of biomarkers in bioanalytical LC-MS or LC-MS/MS, firmly grasps the ideals of using genuine unadulterated matrix together with genuine unlabeled analyte reference materials, obviating the need to prove parallelism in this context. The manner in which the endogenous level is removed from an OA approach calibration line, which is initially constructed as per standard addition, is to simply remove the intercept from the calculated regression equation and use only the slope. In essence, translating the line to intercept the Origin while maintaining the slope thereby eliminating the endogenous level from calibrants, and interpolation of study samples' responses can then take place through the OA approach line, giving reliable calculated concentrations. Furthermore, in the same way that the concentration-intercept in standard addition is a reliable measurement of the endogenous, so the origin becomes a reliable anchor point in OA approach, addressing concern over the semi-quantitative region of the graph below the peak area response of the lowest concentration calibrants.

All documents referred to in this specification are herein incorporated by reference. Various modifications and variations to the described embodiments of the present disclosure will be apparent to those skilled in the art without departing from the scope of the invention. Although the present disclosure has been described in connection with specific preferred embodiments, it should be understood that the systems and methods of the present disclosure as claimed should not be unduly limited to such specific embodiments.

What is claimed is:

1. A method for quantifying an endogenous analyte of interest in a sample, comprising:
obtaining a sample;
spiking the sample with the endogenous analyte of interest;
chromatographically separating the endogenous analyte of interest from other components in the sample;
detecting the endogenous analyte of interest from the sample using mass spectrometry;
calculating a linear regression using the spiked sample of the endogenous analyte of interest;
adjusting a concentration-intercept of the linear regression; and
determining a quantity of the endogenous analyte of interest using the adjusted concentration-intercept.

2. The method of claim 1, wherein the sample is a biological sample.

3. The method of claim 2, wherein the biological sample comprises one of blood, serum, plasma, sputum, saliva, cerebral spinal fluid, or urine.

4. The method of claim 1, wherein the endogenous analyte of interest comprises amino acids, nucleic acids, proteins, enzymes, sugars, ketones, carbohydrates, and biochemical molecules that are associated with metabolism.

5. The method of claim 4, wherein the endogenous analyte of interest further comprises tryptophan and kynurenine.

6. The method of claim 1, wherein chromatographically separating the endogenous analyte of interest is performed by high-pressure liquid chromatography (HPLC), liquid chromatography-mass spectrometry (LC-MS), high turbulence liquid chromatography (HTLC), ultra-performance liquid chromatography (UPLC), liquid chromatography tandem mass spectrometry (LC-MS/MS), and/or ultra-high pressure liquid chromatography (UHPLC).

7. The method of claim 6, wherein chromatographically separating the endogenous analyte of interest further comprises separating analytes using an organic-aqueous solvent mixture to generate a gradient experimentally in which the analyte of interest becomes soluble.

8. The method of claim 6, wherein chromatographically separating the endogenous analyte of interest further comprises separating analytes using a reverse-phase liquid chromatography column.

9. The method of claim 6, wherein liquid chromatography tandem mass spectrometry (LC-MS/MS) comprises the steps of: (i) generating a precursor ion of the endogenous analyte of interest; (ii) generating one or more fragment ions of the precursor ion; and (ii) detecting the presence or amount of the precursor ion generated in step (i) and/or the at least one or more fragment ions generated in step (ii), or both, and relating the detected ions to the presence or amount of the analyte of interest in the sample.

10. The method of claim 9, wherein liquid chromatography tandem mass spectrometry (LC-MS/MS) comprises selecting one or more fragment ions for quantitation of the analyte of interest and selecting one or more additional qualifier fragment ions as a qualitative standard.

11. A system for identifying and quantifying an endogenous analyte of interest in a sample, comprising:
a station for chromatographically separating the endogenous analyte of interest from other components in the sample;
a station for detecting the presence of the endogenous analyte of interest;
a station for analyzing the chromatographically separated endogenous analyte of interest by mass spectrometry to determine a presence or amount of the endogenous analyte of interest in the sample; and
a report for the endogenous analyte's presence and concentration as identified by ion fragmentation and absorption spectra.

12. The system of claim 11, wherein chromatographically separating the endogenous analyte of interest further comprises separating analytes using an organic-aqueous solvent mixture to generate a gradient experimentally in which the analyte of interest becomes soluble.

13. The system of claim 11, wherein chromatographically separating the endogenous analyte of interest further comprises separating analytes using a reverse-phase liquid chromatography column.

14. A computer-program product tangibly embodied in a non-transitory machine-readable storage medium including instructions configured to cause one or more data processors to perform processing comprising: (a) separating an endogenous analyte of interest from the sample; (b) detecting the endogenous analyte of interest; and (c) detecting an amount of the endogenous analyte of interest separated from the sample.
